SEQUENCE LISTING (14 sequences)

SEQ ID NO: 1 - DNA, 1235 bp, murine
  CDS (161)..(799)
cgaggctcaagctccgggcggattctgcgtgccgctctcgctccttggggtctgttggccggcctgtgccacccggacgcccggctcactgcctctgtctcccccatcagcgcagccccggacgctatggcccacccctccagctggcccctcgagtaggatggtagcacgcaaccaggtggcagccgacaatgcgatctccccggcagcagagccccgacggcggtcagagccctcctcgtcctcgtcttcgtcctcgccagcggcccccgtgcgtccccggccctgcccggcggtcccagccccagcccctggcgacactcacttccgcaccttccgctcccactccgattaccggcgcatcacgcggaccagcgcgctcctggacgcctgcggcttctattggggacccctgagcgtgcacggggcgcacgagcggctgcgtgccgagcccgtgggcaccttcttggtgcgcgacagtcgtcaacggaactgcttcttcgcgctcagcgtgaagatggcttcgggccccacgagcatccgcgtgcacttccaggccggccgcttccacttggacggcagccgcgagaccttcgactgccttttcgagctgctggagcactacgtggcggcgccgcgccgcatgttgggggccccgctgcgccagcgccgcgtgcggccgctgcaggagctgtgtcgccagcgcatcgtggccgccgtgggtcgcgagaacctggcgcgcatccctcttaacccggtactccgtgactacctgagttccttccccttccagatctgaccggctgccgctgtgccgcagcattaagtgggggcgccttattatttcttattattaattattattatttttctggaaccacgtgggagccctccccgcctgggtcggagggagtggttgtggagggtgagatgcctcccacttctggctggagacctcatcccacctctcaggggtgggggtgctcccctcctggtgctccctccgggtcccccctggttgtagcagcttgtgtctggggccaggacctgaattccactcctacctctccatgtttacatattcccagtatctttgcacaaaccaggggtcggggagggtctctggcttcatttttctgctgtgcagaatatcctattttatatttttacagccagtttaggtaataaactttattatgaaagtttttttttaaaagaaaaaaaaaaaaaaaaaa

SEQ ID NO: 2 - protein, 212 aa, murine
MVARNQVAADNAISPAAEPRRRSEPSSSSSSSSPAAPVRPRPCPAVPAPAPGDTHFRTFRSHSDYRRITRTSALLDACGFYWGPLSVHGAHERLRAEPVGTFLVRDSRQRNCFFALSVKMASGPTSIRVHFQAGRFHLDGSRETFDCLFELLEHYVAAPRRMLGAPLRQRRVRPLQELCRQRIVAAVGRENLARIPLNPVLRDYLSSFPFQI

SEQ ID NO: 3 - DNA, 2187 bp, murine
  CDS (18)..(695)
cgctggctccgtgcgccatggtcacccacagcaagtttcccgccgccgggatgagccgccccctggacaccagcctgcgcctcaagaccttcagctccaaaagcgagtaccagctggtggtgaacgccgtgcgcaagctgcaggagagcggattctactggagcgccgtgaccggcggcgaggcgaacctgctgctcagcgccgagcccgcgggcacctttcttatccgcgacagctcggaccagcgccacttcttcacgttgagcgtcaagacccagtcggggaccaagaacctacgcatccagtgtgaggggggcagcttttcgctgcagagtgacccccgaagcacgcagccagttccccgcttcgactgtgtactcaagctggtgcaccactacatgccgcctccagggaccccctccttttctttgccacccacggaaccctcgtccgaagttccggagcagccacctgcccaggcactccccgggagtacccccaagagagcttactacatctattctgggggcgagaagattccgctggtactgagccgacctctctcctccaacgtggccaccctccagcatctttgtcggaagactgtcaacggccacctggactcctatgagaaagtgacccagctgcctggacccattcgggagttcctggatcagtatgatgctccactttaaggagcaaaagggtcagaggggggcctgggtcggtcggtcgcctctcctccgaggcacatggcacaagcacaaaaatccagccccaacggtcggtagctcccagtgagccaggggcagattggcttcttcctcaggccctccactcccgcagagtagagctggcaggacctggaattcgtctgaggggagggggagctgccacctgctttcccccctcccccagctccagcttctttcaagtggagccagccggcctggcctggtgggacaatacctttgacaagcggactctcccctccccttcctccacaccccctctgcttcccaagggaggtggggacacctccaagtgttgaacttagaactgcaaggggaatcttcaaactttcccgctggaacttgtttgcgctttgatttggtttgatcaagagcaggcacctgggggaaggatggaagagaaaagggtgtgtgaagggtttttatgctggccaaagaaataaccactcccactgcccaacctaggtgaggagtggtggctcctggctctggggagagtggcaaggggtgacctgaagagagctatactggtgccaggctcctctccatggggcagctaatgaaacctcgcagatcccttgcaccccagaaccctccccgttgtgaagaggcagtagcatttagaagggagacagatgaggctggtgagctggccgccttttccaacaccgaagggaggcagatcaacagatgagccatcttggagcccaggtttcccctggagcagatggagggttctgctttgtctctcctatgtggggctaggagactcgccttaaatgccctctgtcccagggatggggattggcacacaaggagccaaacacagccaataggcagagagttgagggattcacccaggtggctacaggccaggggaagtggctgcaggggagagacccagtcactccaggagactcctgagttaacactgggaagacattggccagtcctagtcatctctcggtcagtaggtccgagagcttccaggccctgcacagccctcctttctcacctggggggaggcaggaggtgatggagaagccttcccatgccgctcacaggggcctcacgggaatgcagcagccatgcaattacctggaactggtcctgtgttggggagaaacaagttttctgaagtcaggtatggggctgggtggggcagctgtgtgttggggtggcttttttctctctgttttgaataatgtttacaatttgcctcaatcacttttataaaaatccacctccagcccgcccctctccccactcaggccttcgaggctgtctgaagatgcttgaaaaactcaaccaaatcccagttcaactcagactttgcacatatatttatatttatactcagaaaagaaacatttcagtaatttataataaaagagcactattttttaatgaaaaaaaaaaaaaaaaaaaaaaaaaa

SEQ ID NO: 4 - protein, 225 aa, murine
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGTPSFSLPPTEPSSEVPEQPPAQALPGSTPKRAYYIYSGGEKIPLVLSRPLSSNVATLQHLCRKTVNGHLDSYEKVTQLPGPIREFLDQYDAPL

SEQ ID NO: 5 - DNA, 1094 bp, human
ctccggctggccccttctgtaggatggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatttgaccggcagcgcccgccgtgcacgcagcattaactgggatgccgtgttattttgttattacttgcctggaaccatgtgggtaccctccccggcctgggttggagggagcggatgggtgtaggggcgaggcgcctcccgccctcggctggagacgaggccgcagaccccttctcacctcttgagggggtcctccccctcctggtgctccctctgggtccccctggttgttgtagcagcttaactgtatctggagccaggacctgaactcgcacctcctacctcttcatgtttacatatacccagtatctttgcacaaaccaggggttgggggagggtctctggctttatttttctgctgtgcagaatcctattttatattttttaaagtcagtttaggtaataaactttattatgaaagtttttttttttaaaaaaaa

SEQ ID NO: 6 - protein, 211 aa, human
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 7 - DNA, 2807 bp, murine
ggaaaccgaggcggggagaccaggaggccttggcctcagagcttcagagtcgcgtggcagcaaacagagaaacctgtagagggcagtgtgcgtcacttagctcagggaagctgcacgcgaaactcacccgccttcattcataaacatcgtcagctaggcacctactcctgggctttcaggacaaactgaatcacgaaaccacagtgtccttaaaataggtctgaccgcctgaatccctggccaaggtgtgtacggggcatgggagcccttgtgcagagatgcttgcaggagccttgaggggctctgtaagacagaggctaggaagacaaagttgggggctacagcttcttgtcctgcccggggcctcagtttcttcggttgcccacgtaggagtgcagagagtccagcccctggggacccaacccaaccccgcccagtttccgaggaactcgtccgggagcgggggcgcccctcccgcaccgccttaggcttcctttgaagcctctgcggtcaggccaccgcttcctgggaagcccaagccaaggccaggccgagtggccaacgggaggggcccgcgcgcgattctggaggagggcggcggccccacaggtctccagggctggctagccgggctcctagagcggagactgccaaggccttcgggtcctgggcaggaaggatcctggcagggaggagttgcttggggggtgggggggaaaggctccaggcgcggtggagctctgaccaggagaatgcacacactcggaggggaggaggcgtgtcagccccaagctagcatcccacccggggagcagcgatgtggggcgaaggtagccagagcaaaagagcaggcaccaggtgacacgaaacagaagattccgggtagagccagaaccccagaagtcccattcagggaaggtgcgaggcgagaacgagttaggtggaccctctccaggggcagccaaagaaatctaaagagaacccgaaggacttgccggaaagagaaaccgaaagcggcggtgggcgggatcggtgggcggggcctccctggtttaagagcttgatgcaggggcgggcagcagcagagagaactgcggccgtggcagcggcacggctcccggccccggagcatgcgcgacagcagccccggaacccccagccgcggcgccccgcgtcccgccgccaggtgagccgaggcagctgcgaaggagcaggcgggaggggatgggaggaaggggagcagagcctggcaggactatcctcgcagactgcatggcggggtcgtggatgctatgcctctggcgcccgccccaccggctggcccaggcggcccctcgcgcgcgcggggcgccgtcagcccctcctctccggccctgagcccggatcgtccgcccgggttccagttcccggcgtggccagtaggcggcaaccgcgaggcggcaagccacccagcggggacggcctggagtcgggcccctctccacgcccccttctccacgcgcgcggggaggcagggctccaccgccagtctggaagggttccacatacaggaacggcctacttcgcagatgagcccaccgaggctcaggctccgggcggattctgcgtgtcaccctcgctccttggggtccgctggccggcctgtgccacccggacgcccggttcactgcctctgtctcccccatcagcgcagccccggacgctatggcccacccctccagctggcccctcgagtaggatggtagcacgtaaccaggtggaagccgacaatgcgatctccccggcatcagagccccgacggcggccagagccatcctcgtcctcgtcttcgtcctcgccggcggccccggcgcgtccccggccctgcccggtggtcccggccccggctccgggcgacactcacttccgcaccttccgctcccactctgattaccggcgcatcacgcggaccagcgctctcctggacgcctgcggcttctactggggacccctgagcgtgcatggggcgcacgaacggctgcgttccgaacccgtgggcaccttcttggtgcgcgacagtcgccagcggaactgcttcttcgcgctcagcgtgaagatggcttcgggccccacgagcattcgtgtgcacttccaggccggccgcttccacctggacggcaaccgcgagaccttcgactgcctcttcgagctgctggagcactacgtggcggcgccgcgccgcatgttgggggccccactgcgccagcgccgcgtgcggccgctgcaggagctgtgtcgccagcgcatcgtggccgccgtgggtcgcgagaacctggcacgcatccctcttaacccggtactccgtgactacctgagttccttccccttccagatctgaccggctgccgccgtgcccgcagaattaagtgggagcgccttattatttcttattattaattattattatttttctggaaccacgtgggagccctccccgcctaggtcggagggagtgggtgtggagggtgagatccctcccacttctggctggagaccttatcccgcctctcggggggcctcccctcctggtgctccctcccggtccccctggttgtagcagcttgtgtctggggccaggacctgaactccacgcctacctctccatgtttacatgttcccagtatctttgcacaaaccaggggtgggggagggtctctggcttcatttttctgctgtgcagaatattctattttatatttttacatccagtttagataataaactttattatgaaagtttttttttttaaagaaacaaagatttctaga

SEQ ID NO: 8 - protein, 212 aa, murine
MVARNQVEADNAISPASEPRRRPEPSSSSSSSSPAAPARPRPCPVVPAPAPGDTHFRTFRSHSDYRRITRTSALLDACGFYWGPLSVHGAHERLRSEPVGTFLVRDSRQRNCFFALSVKMASGPTSIRVHFQAGRFHLDGNRETFDCLFELLEHYVAAPRRMLGAPLRQRRVRPLQELCRQRIVAAVGRENLARIPLNPVLRDYLSSFPFQI

SEQ ID NO: 9 - DNA, 35 bp, primer
atatctcgaggccaccatggtagcacgcaaccagg

SEQ ID NO: 10 - DNA, 30 bp, primer
atataagctttcagatctggaaggggaagg

SEQ ID NO: 11 - DNA, 35 bp, primer
atatgcggccgcgccaccatgaccctgcggtgcct

SEQ ID NO: 12 - DNA, 35 bp, primer; identical to SEQ ID NO: 11

SEQ ID NO: 13 - DNA, 35 bp, primer; identical to SEQ ID NO: 11

SEQ ID NO: 14 - DNA, 35 bp, primer; identical to SEQ ID NO: 11

(19) United States
(12) Patent Application Publication
(10) Pub. No.: US 2004/0072752 A1
(43) Pub. Date: Apr. 15, 2004

(54) Modulating cytokine or hormone signalling in an animal comprising up-regulating the expression of socs sequence in the animal

(76) Inventors: Andrew Nash, Victoria (AU); Pino Maccarone, Victoria (AU); Paul Egan, Victoria (AU); Ian Wicks, Victoria (AU)

Correspondence Address: Leopold Presser, Scully Scott Murphy & Presser, 400 Garden City Plaza, Garden City, NY, 11530, US

(21) Appl. No.: 10/398,863

(22) PCT Filed: Oct. 9, 2001
(86) PCT No.: PCT/AU01/01272

(30) Foreign Application Priority Data
Oct. 9, 2000 (AU) PR 0647
Dec. 7, 2000 (AU) PR 1942

(51) Int. Cl. A61K 38/23

(52) U.S. Cl. 514/12

(57) ABSTRACT

The present invention relates generally to a method for the treatment and/or prophylaxis of conditions arising from or otherwise associated with aberrations in hormone signalling. More particularly, the present invention contemplates a method for the treatment and/or prophylaxis of conditions, the amelioration of symptoms of which, are facilitated by an over-expression of a gene encoding a suppressor of cytokine signalling molecule. The present invention further contemplates agents useful for the prophylaxis and/or treatment of such renditions in mammals including humans.

### FIELD OF THE INVENTION

The present invention relates generally to a method for the treatment and/or prophylaxis of conditions arising from or otherwise associated with aberrations in hormone signalling.

More particularly, the present invention contemplates a method for the treatment and/or prophylaxis of conditions, the amelioration of symptoms of which are facilitated by an over-expression of a gene encoding a suppressor of cytokine signalling molecule. The present invention further contemplates agents useful for the prophylaxis and/or treatment of such conditions in mammals including humans.

### BACKGROUND OF THE INVENTION

Bibliographic details of the publications numerically referred to in this specification are collected at the end of the description.

Reference to any prior art in this specification is not, and should not be taken as, au acknowledgment or any form of suggestion that this prior art forms part of the common general knowledge in Australia or any other country.

The gene encoding Suppressor of Cytokine Signaling-1 (SOCS-1), the SOCS protein family prototype, was discovered in a functional genetic screen designed to identify inhibitors of cytokine signalling. Comparison to existing sequences on genetic databases identified a number of additional proteins that could be grouped into a “SOCS protein family” on the basis of homology within a novel COOH-terminal ‘SOCS-box’ sequence motif. Proteins containing the SOCS-box could be further divided into sub-families on the basis of additional protein sequence motifs including, for example, SH2 domains (SOCS1-7), WD40 repeats (WSB1, 2), ankyrin repeats (ASB1-3) and a SPRY domain (SSB1-3). Subsequent analysis has revealed tat SOCS-1 and other SOCS family members, most notably those which incorporate an SH2 domain, represent the key components of a classic negative feedback loop that regulates cytokine signalling. SOCS protein expression is induced by cytokine signaling and SOCS proteins interact with components of that process to tub signaling off.

SOCS-1, which inhibits the in vitro activity of a variety of cytokines including IL-6, LIF, and type 19 interferons, binds directly to, and inhibits the action of, Janus kinses (JAKs). Published analysis indicates that this activity against JAKs may be mediated by three distinct functional domains within SOCS-1: the SH2 domain and preceding 12 ammo acids (extended SH2 subdomain) of SOCS-1 are required for binding to the phosphorylated (Y1007) activation loop of JAK2; an additional 12 N-terminal amino acids (kinase inhibitory region) of SOCS-1 contribute to high affinity binding to the JAK2 tyrosine e dowry and are required for the inhibition of JAK2 activity; and the SOCS-box has been found to mediate the inter on of SOCS proteins with elongin B and elongin C, intracellular proteins responsible for targeting proteins for degradation within the cell.

In addition to inhibiting the activity of cytokines that signal through the JAK/STAT pathway, SOCS-1 has also been reported to inhibit TNFα activities such as induction of cell death (1). Although the mechanism for this activity remains unclear, there is some evidence to suggest that SOCS-1 regulates the activity of p38 MAP kinase which in turn may act as a survival factor in TNF treated cells.

SOCS-3 has also been demonstrated to inhibit the in vitro activity of LIF and IL-6, however, in contrast to SOCS-1, it does not appear to bind directly to JAKs. Structure-function studies have identified an interaction between SOCS-3 and he cytoplasmic domain of shared receptor component gp130. In particular a single peptide representing the amino acid stretch 750764 of gp130 and centred around the phosphorylated tyrosine residue 757 (pY757) is able to bind to the SOCS-3 protein with high affinity (dd-42 nm). Thus, SOCS proteins appear to inhibit cytokine signalling by at least two mechanisms: they are able to bind to, and inhibit the activity of signalling intermediates activated following receptor oligermerization (e.g. JAKs) or they interact with receptor components (e.g. gp130) to inhibit the phosphorlyation and activation of downstream substrates.

Cytokines are key mediators of a number of severe and debilitating diseases. For example, a number of cytokines including IL-1, IL-6, TNFα, GM-CSF and type I/II interferons are central to the pathophysiology of both acute and chronic inflammatory disease. This is reflected in the development and marketing of new therapeutic strategies which focus on inhibition of cytokine action. For example, specific antagonists of TNFα (monoclonal antibodies, soluble receptors) are now used successfully in the treatment of rheumatoid arthritis and Chrones disease.

As potent negative regulators of cytokine signalling SOCS proteins provide for a new approach to the treatment of cytokine mediated disease such as rheumatoid arthritis. Targeted over-expression of SOCS proteins (i.e. SOCS proteins as gene therapeutics) should turn off cytokine signalling and ameliorate cytokine-mediated disease. Rheumatoid arthritis represents a useful example. When over-expressed, SOCS-1 has been demonstrated to interact with and inhibit the activity of JAKs. JAK activation and subsequent action represents an important downstream event in signalling through both IL-6 and GM-CSF receptors. Furthermore SOCS-1 has also been demonstrated to be a potent antagonist of TNFα mediated activities. In work leading up to the present invention, the inventors reasoned that over-expression of SOCS-1 could be expected to interfere in IL-6, GM-CSF and TNF signalling, all key mediators of rheurmatoid arthritis.

For SOCS therapeutics to be effective, it is likely that they will need to be expressed at a high level such as being over-expressed in the majority of target cells within a pathological lesion Gene based therapies clearly represent the best way to achieve this, with viral vectors such as adenovirus, adeno-associated virus (AAV) and retrovirus likely to represent the delivery mechanism of choice.

### SUMMARY OF THE INVENTION

Nucleotide and amino acid sequences are referred to by a sequence identifier number (SEQ ID NO:). The SEQ ED NOs: correspond numerically to the sequence identifiers <400>1, <400>2, etc. A sequence listing is provided after the claims.

Throughout this specification, unless the context requires otherwise, the word “comprise”, or variations such as “comprises” or “comiprising”, will be understood to imply the inclusion of a stated element or integer or group of elements or integers but not the exclusion of any other element or integer or group of elements or integers.

The present invention is predicated in part on the use of genetic therapeutic protocols to increase, enhance or otherwise facilitate expression of nucleotide sequences encoding a SOCS molecule in a cell. Overexpression of such nucleotide sequences thereby elevates levels of the SOCS protein or other expression products (e.g. mRNA or spliced out introns from mRNA encoded by genomic DNA). The “over-expression” in this context means, in one particular embodiment, a level of expression statistically greater than a standardized normal control. However, the present invention also contemplates maintenance of normal expression levels. The “level” of expression may readily be determined by, for example, nuclear run-on analysis or determination of SOCS protein levels amongst other methods.

Accordingly, one aspect of the present invention contemplates a method for modulating cytokine or hormone signalling in an animal, said method comprising up-regulating expression of a genetic sequence encoding a SOCS protein or its derivative or homolog in said animal.

Another aspect of the present invention provides a method of modulating cytokine or hormone signalling in an animal and in particular a human, said method comprising up-regulating expression of a genetic sequence encoding a SOCS protein in said animal and wherein said SOCS protein comprises a protein molecule interacting region such as but not limited to an SH2 domain, WD-40 repeats and/or ankyrin repeats, N terminal of a SOCS box, wherein said SOCS box comprises the amino acid sequence:

X₁X₂X₃X₄X₅X₇X₈X₉X₁₀X₁₁X₁₂ X₁₃X₁₄X₁₅X₁₆[Xᵢ]ₙX₁₇X₁₈X₁₉X₂₀X₂₁X₂₂X₂₃[Xⱼ] X₂₄X₂₅X₂₆X₂₇X₂₈

wherein:

X₁ is L, I, V, M, A or P;

X₂ is any amino acid residue;

X₃ is P, T or S;

X₄ is L, I, V, M, A or P;

X₅ is any amino acid;

X₆ is any amino acid;

X₇ is L, I, V, M, A, F, Y or W;

X₈ is C, T or S;

X₉ is R, K or H;

X₁₀ is any amino acid;

X₁₁ is any amino acid;

X₁₂ is L, I, V, M, A or P;

X₁₃ is any amino acid;

X₁₄ is any amino acid;

X₁₅ is any amino acid;

X₁₆ is L, I, V, M, A, P, G, C, T or S;

[Xᵢ]ₙ is a sequence of n amino acids wherein u is from 1 to 50 amino acids and wherein the sequence Xᵢ may comprise the same or different amino acids selected from any amino acid residue;

X₁₇ is L, I, V, M, A or P;

X₁₈ is any amino acid;

X₁₉ is any amino acid;

X₂₀ is L, I, V, M, A or P;

X₂₁ is P;

X₂₂ is 1L, I, V, M, A, P or G;

[Xⱼ]ₙ is a sequence of n amino acids wherein n is from 0 to 50 amino acids and wherein the Xᵢ may comprise the same or different amino acids selected from any amino acid residue,

X₂₄ is L, V, A or P;

X₂₅ is any amino acid;

X₂₆ is any amino acid,

X₂₇ is Y or F;

X₂₈ is L, I, V, M, A or P.

Still another aspect of the present invention contemplates a method for controlling cytokine or hormone signalling, such as pro-inflammatory cytokine signalling (i.e. 16, GM-CSF, TNFα), in an animal such as a human or livestock animal, said method comprising modulating expression of a genetic sequence encoding a SOCS protein comprising a SOCS box and a protein molecule interacting region N-terminal of said SOCS box wherein said SOCS box comprises the amino acid sequence;

X₁X₂X₃X₄X₅X₆X₇X₈X₉X₁₀X₁₁X₁₂X₁₃X₁₄X₁₅X₁₆[Xᵢ]ₙX₁₇X₁₈X₁₉X₂₀X₂₁X₂₂X₂₃[Xⱼ]ₙX₂₄X₂₅X₂₆X₂₇X₂₈

wherein:

X₁ is L, I, V, M, A or P;

X₂ is any amino acid residue;

X₃ is P, T or S;

X₄ is L, I, V, M, A or P;

X₅ is any amino acid;

X₆ is any amino acid;

X₇ is L, I, V, M, A, F, Y or W;

X₈ is C, T or S;

X₉ is R, K or H;

X₁₀ is amino acid;

X₁₁ is any amino acid;

X₁₂ is L, V, M, A or P;

X₁₃ is any amino acid;

X₁₄ is any amino acid;

X₁₅ is any amino acid;

X₁₆ is L, I, V, M, A, P, G, C, T or S;

[Xᵢ]ₙ is a sequence of n amino acids wherein n is from 1 to 50 amino acids and wherein the sequence Xᵢ may comprise the same or different amino acids selected from any amino acid residue;

X₁₇ is L, I, V, M, A or P;

X₁₈ is any amino acid;

X₁₉ is any amino acid;

X₂₀ is L, I, V, A or P;

X₂₁ is P;

X₂₂ is L, I, V, M, A, P or G;

X₂₃ is P or N;

[X₂₄]ₙ is a sequence of n amino acids wherein n is from 0 to 50 amino acids and wherein the Xⱼ may comprise the same or different amino acids selected from any amino acid residue;

X₂₄ is L, I, V, M, A or P;

X₂₅ is any amino acid;

X₂₆ is amino acid;

X₂₇ is Y or F;

X₂₈ is L, I, V, M, A or P.

Yet another aspect of the present invention contemplates a method for controlling cytokine or hormone signaling in an animal such as human or livestock animal, said method comprising administering to said animal a genetic molecule encoding a SOCS protein for a time and under conditions sufficient to modulate growth hormone signaling.

Another aspect of the present invention contemplates a method for the treatment of cytokine-mediated disease in an animal, said method comprising modulating cytokine or hormone signalling in an animal by up-regulating the expression of a genetic sequence encoding a SOCS protein or its derivative or homologue in said animal.

In a preferred embodiment, the SOCS gene is expressed at a high level such as being overexpressed.

A summary of sequence identifiers used throughout the subject specification is provided below.

### SUMMARY OF SEQUENCE IDENTIFIERS

| SEQUENCE ID NO: | DESCRIPTION |
|---|---|
| 1 | Mouse SOCS-1 (nucleotide) |
| 2 | Mouse SOCS-1 (amino acid) |
| 3 | Mouse SOCS-3 (nucleotide) |
| 4 | Mouse SOCS-3 (amino acid) |
| 5 | Human SOCS-1 (nucleotide) |
| 6 | Human SOCS-1 (amino acid) |
| 7 | Rat SOCS-1 (nucleotide) |
| S | Rat SOCS-1 (amino acid) |
| 9 | Primer |
| 10 | Primer |
| 11 | Primer |
| 12 | Primer |
| 13 | Primer |
| 14 | Primer |

### BRIEF DESCRIPTION OF THE FIGURES

FIG. 1 is a graphical representation of SOCS-1^{+/+} IFN-γ^{-/-}mice (▪) compared to SOCS-1^{+/+} IFN-γ^{-/-} (□) mice following injection of BSA and L-1 subcutaneously to knee joints in three daily injections. A histological score was measured in oxodate, synovitis, pannus, cartilage and bone.

### DETAILED DESCRIPTION OF THE PREFERRED EMBODIMENTS

One aspect of the present invention contemplates a method for modulating cytokine or hormone signalling in an animal, said method comprising up-regulating expression of a genetic sequence encoding a SOCS protein or its derivative or homolog in said animal.

Reference herein to “SOCS” encompasses any or all members of the SOCS family. Specific SOCS molecules may be defined numerically such as, for example, SOCS-1, SOCS-2 and SOCS-3. The species from which the SOCS has been obtained may be indicated by a preface of single letter abbreviation where “h” is human, “m” is mouse and “r” is rat. Accordingly, “mSOCS-2”, for ale, is a specific SOCS from a murine animal. Reference herein to “SOCS” is not to imply that the protein solely suppresses cytokine-mediated signal transduction, as the molecule may modulate other effector-mediated signal transductions such as by hormones or other endogenous or exogenous molecules, antigen, microbes and microbial products, viruses or components thereof, ions, hormones and parasites. The to “modulates” encompasses up-regulation as well as at least maintenance of particular levels. Preferably, the expression is up-regulated. Reference herein to “murine” includes both mouse and rat.

Reference herein to a “hormone” includes protein hormones as well as non-proteinaceous hormones. One particularly useful hormone is growth hormone. Another useful hormones are insulin-like growth factor I (IGF-I) and prolactin. A cytokine refers to any cytokine or cytokine-like molecule such as interleukin (e.g. IL-1, IL-6), tumour necrosis factor (e.g. TNFα), a colony stimulating factor (e.g. GM-CSF) or an interferon.

An “animal” is preferably a mammal such as but not limited to a human, primate, livestock animal (e.g. sheep, cow, pig, horse, donkey), laboratory test animal (e.g. rabbit, mouse, rat, guinea pig), companion animal (e.g. cat, dog) or captive wild animal. The annual may be in the form of an animal model. Useful animals for this purpose are laboratory test animals. Genetically modifying livestock animals is useful in assisting in food production The preferred animal is a human, primate animal or laboratory test animal The most preferred animal is a human.

Reference herein to “SOCS” includes a protein comprising a SOCS box in its C-terminal region comprising the amino acid sequence:

X₁X₂X₃X₄X₅X₆X₇X₈X₉X₁₀X₁₁X₁₂X₁₃X₁₄X₁₅X₁₆[Xᵢ]ₙX₁₇X₁₈X₁₉X₂₀X₂₁X₂₂X₂₃[Xⱼ]ₙX₂₄X₂₅X₂₆X₂₇X₂₈

wherein:

X₁ is L, I, V, M, A or P;

X₂ is any amino acid residue;

X₃ is P, T or S;

X₄ is L, I, V, M, A or P;

X₅ is any amino acid;

X₆ is any amino acid;

X₇ is L, I, V, M, A, F, Y or W;

X₈ is C, T or S;

X₉ is R, K or I;

X₁₀ is any amino acid;

X₁₁ is any amino acid;

X₁₂ is L, I, V, M, A or P;

X₁₃ is any amino acid;

X₁₄ is any amino acid;

X₁₅ is any amino acid;

X₁₆ is L, I, V, M, A, P, G, C, T or S;

[Xᵢ]ₙ is a sequence of n amino acids wherein n is from 1 to 50 amino acids and wherein the sequence Xᵢ may comprise the same or different amino acids selected from any in acid residue;

X₁₇ is L, I, V, M, A or P;

X₁₈ is amino acid;

X₁₉ is any amino acid;

X₂₀ is L, V, M, A or P;

X₂₁ is P;

X₂₂ is L, I, V, W, A, P or G;

X₂₃ is P or N,

[Xⱼ]ₙ is a sequence of n amino acids wherein n is from 0 to 50 amino acids and wherein the Xⱼ may comprise the same or different amino acids selected from any amino acid residue;

X₂₄ is L, I, V, M, A or P;

X₂₅ is any amino acid;

X₂₆ is any amino acid;

X₂₇ is Y or F;

X₂₈ is L, I, V, M, A or P.

The SOCS protein also comprises a protein:molecule interacting region such as but not limited to one or more of an SH2 domain, WD-40 repeats and/or ankyrin repeats, N-terminal of the SOCS box.

In an important aspect, the present invention contemplates un-regulating expression of a nucleotide sequence encoding a SOCS protein in the treatment of inflammatory diseases such as rheumatic arthritis.

Another aspect of the present invention provides a method of modulating cytokine or hormone signalling in an animal and in particular a human, said method comprising up-regulating expression of a genetic sequence encoding a SOCS protein in said anima and wherein said SOCS protein comprises a protein:molecule interacting region such as but not limited to an SE domain, WD-40 repeats and/or ankyrin repeats, N terminal of a SOCS box, wherein said SOCS box comprises the amino acid sequence:

X₁X₂X₃X₄X₅X₆X₇X₈X₉X₁₀X₁₁X₁₂X₁₃X₁₄X₁₅X₁₆[Xᵢ]ₙX₁₇X₁₈X₁₉X₂₀X₂₁X₂₂X₂₃[Xⱼ]ₙX₂₄X₂₆X₂₇X₂₈

wherein:

X₁ is L, I, V, M, A or P;

X₂ is any amino acid residue;

X₃ is P, T or S;

X₄ is L, I, V, M, A or P;

X₅ is any amino acid;

X₆ is any amino acid;

X₇ is L, I, M, A, F, Y or W;

X₈ is C, T or S;

X₉ is R, K or H;

X₁₀ is any amino acid;

X₁₁ is any amino acid;

X₁₂ is L, I, V, M, A or P;

X₁₃ is any amino acid;

X₁₄ is any amino acid;

X₁₅ is any amino acid;

X₁₆ is L, I, V, M, A, P, G, T or S;

[Xᵢ]ₙ is a sequence of n amino acids wherein n is from 1 to 50 amino acids and wherein the sequence Xᵢ may comprise the same or different amino acids selected from any amino acid residue;

X₁₇ is L, V, M, A or P;

X₁₈ is any amino acid;

X₁₉ is any amino acid;

X₂₀ is L, I, V, M, A or P;

X₂₁ is P;

X₂₂ is L, I, V, M A, P or G;

X₂₃ is P or N;

[Xⱼ]ₙ is a sequence of n amino acids wherein n is from 0 to 50 amino acids and wherein the Xⱼ; may comprise the same or different amino acids selected from any amino acid residue;

X₂₄ is L, I, V, M, A or P;

X₂₅ is any amino acid;

X₂₆ is any amino acid;

X₂₇ is Y or F;

X₂₈ is L, I, V, M, A or P.

The present invention extends to any SOCS molecule such as those disclosed in International Patent Application No. PCT/AU99/00729 [WO 98/20023] which is incorporated herein by reference. However, in a particularly preferred embodiment, the present invention is directed to manipulating levels of SOCS-1, which murine form (mSOCS-1) comprises the nucleotide and corresponding amino acid sequence as set forth in SEQ ID NO:1 and SEQ ID NO:2, respectively. The present invention is hereinafter described with reference to murine SOCS-1 (mSOCS-1), however, this is done with the understanding that the present invention encompasses the manipulation of levels of any SOCS molecule, such as but not limited to films SOCS-2 (hSOCS-2). Reference herein to a “SOCS” molecule such as SOCS-1 includes any mutants thereof such as functional mutants. An example of a mutant is a single or multiple amino acid substitution, addition and/or deletion or truncation to the SOCS molecule or its corresponding DNA or RNA Accordingly, another aspect of the present invention contemplates a method for controlling cytokine or hormone signaling such as pro-inflammatory cytokine signalling (i.e. IL-6, GM-CSF, TNFα), in an animal such as a human or livestock animal, said method comprising modulating expression of a genetic sequence encoding a SOCS protein comprising a SOCS box and a protein:molecule interacting region N-terminal of said SOCS box wherein said SOCS box comprises the amino acid sequence:

X₁X₂X₃X₄X₅X₆X₇X₈X₉X₁₀X₁₁X₁₂X₁₃X₁₄X₁₅X₁₆[Xᵢ]ₙX₁₇X₁₈X₁₉X₂₀X₂₁X₂₂X₂₃[Xⱼ]ₙX₂₄X₂₆X₂₇X₂₈

wherein:

X₁ is L, I, V, M, A or P;

X₂ is any amino acid residue;

X₃ is P, T or S;

X₄ is L, I, V, M, A or P;

X₅ is any amino acid;

X₆ is any amino acid;

X₇ is L, I, M, A, F, Y or W;

X₈ is C, T or S;

X₉ is R, K or H;

X₁₀ is any amino acid;

X₁₁ is any amino acid;

X₁₂ is L, I, V, M, A or P;

X₁₃ is any amino acid;

X₁₄ is any amino acid;

X₁₅ is any amino acid;

X₁₆ is L, I, V, M, A, P, G, T or S;

[Xᵢ]ₙ is a sequence of n amino acids wherein n is from 1 to 50 amino acids and wherein the sequence Xᵢ may comprise the same or different amino acids selected from any amino acid residue;

X₁₇ is L, V, M, A or P;

X₁₈ is any amino acid;

X₁₉ is any amino acid;

X₂₀ is L, I, V, M, A or P;

X₂₁ is P;

X₂₂ is L, I, V, M A, P or G;

X₂₃ is P or N;

[Xⱼ]ₙ is a sequence of n amino acids wherein n is from 0 to 50 amino acids and wherein the Xⱼ; may comprise the same or different amino acids selected from any amino acid residue;

X₂₄ is L, I, V, M, A or P;

X₂₅ is any amino acid;

X₂₆ is any amino acid;

X₂₇ is Y or P;

X₂₈ is L, I, M, A or P.

Preferably, the SOCS protein-encoding genetic sequence comprises a nucleotide sequence substantially as set forth in SEQ ID NO: 1, SEQ ID NO:3, SEQ ID NO:5 or SEQ ID NO:7 or a nucleotide sequence having at least 60% similarity hereto or a nucleotide sequence capable of hybridizing to SEQ ID NO:1, SEQ ID NO:3, SEQ ID NO:5 or SEQ ID NO:7 or its complementary form under low stringency conditions at 42° C. Even more preferably, the SOCS protein in a human homolog of the nucleotide sequence set forth in SEQ ID NO:1, SEQ ID NO:3, SEQ ID NO:5 or SEQ ID NO:7.

The term “similarity” as used herein includes exact identity between compared sequences at the nucleotide or amino acid level. Where there is non-identity at the nucleotide level, “similarity” includes differences between sequences which result in different amino acids tat are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similarity” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (2). A detailed discussion of sequence analysis can be found in Unit 19.3 of Ausubel et al. (3).

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a percentage of “sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif. USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions.

Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/V to at least about 30% V/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ 69.3+0.41 (G+C)% (4). However, the Tₘ of a duplex DNA decreases by 1° C. with every increase of 1% in the number of mismatch base pairs (5). Formamide is optional in these hybridization conditions. Accordingly, particularly preferred levels of stringency are defined as follows: low stringency is 6×SSC buffer, 0.1% w/v SDS at 2542° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

Most preferably, an expression vector is administered capable of expressing high levels of a SOCS gene.

Another aspect of the present invention contemplates a method for the treatment of cytokine-mediated disease in an animal, said method comprising modulating cytokine or hormone signalling in an animal by up-regulating the expression of a genetic sequence encoding a SOCS protein or its derivative or homolog in said animal.

In accordance with the this and other aspects of the present invention, the expression of a genetic sequence encoding a SOCS protein is preferably unregulated by the administration to the animal of an expression vector comprising a SOCS gene.

The present invention contemplates a range of derivatives of the SOCS molecule.

A “derivative” includes a part, portion or fragment thereof such as a molecule comprising a single or multiple amino acid substitution, deletion and/or addition. A “homolog” includes a functionally similar molecule from either the same species or another species.

Other derivatives contemplated by the present invention include a range of glycosylation variants from a completely unglycosylated molecule to a modified glycosylated molecule. Altered glycosylation patterns may result from expression of recombinant molecules in different host cells.

The present invention provides, therefore, the genetic control of SOCS levels in animals in the treatment of a range of physiological conditions. Preferably, the level of SOCS protein is increased by the administration of an expression vector comprising the SOCS gene.

Preferably, the expression vector is a viral vector, such as an adenovirus, adeno-associated virus (AAV) or retrovimus, although other vectors, including plasmid-based vectors, are contemplated.

Preferably, the genetic sequence encoding a SOCS protein is the SOCS-i genetic sequence encoding the SOCS-1 protein.

For example, compositions comprising antisense RNA or sense or antisense DNA, ribozymes or sense molecules (for co-suppression) may be administered either locally or systemically to manipulate expression of SOCS genes or translation of SOCS mRNA.

The present invention is further described by the following non-limiting Examples.

### EXAMPLE 1

### Construction of Recombinant Adenovirus for Expression of Selected SOCS Proteins

Recombinant human adenovirus type 5 expressing selected SOCS proteins (for analysis in mouse models of disease mouse SOCS proteins are preferable) are generated following recombination between an adenovirus shuttle vector, into which a SOCS encoding cDNA has been cloned, and a mutant adenovirus. The E1 region has been deleted in the mutant adenovirus rendering it incapable of replication except in a packaging cell line that complements the defect (for example, human 293 cells expressing viral E1A and E1B proteins). Recombination, and subsequent selection of recombinants, can be carried out in the packaging cell line but a bacterial system, referred to as the pAdEasy system is preferred (6)

The pAdEasy system is used to generate recombinant adenovirus expressing murine SOCS proteins by the following means.

Murine SOCS-1 cDNA is amplified by the polymerase chain reaction (PCR), using the following primer set: 5′primer-ATATCTCGAGGCCACCATGGTAGCACGCAACCAGG [SEQ ID NO: 9]; 3′primer-ATATAAGCTTTCAGATCTGGAAGGGGAAGG [SEQ ID NO:10]. The 5′ primer contains a Kozak sequence and XhoI restriction site, while the 3′ primer contains a HindIII restriction site.

Murine SOCS-2 cDNA is amplified by PCR, using the following primer set: 5′ primer-

[SEQ ID NO:11]

3′ primer - ATATGCGGCCGCGCCACCATGACCCTGCGGTGCCT;

[SEQ ID NO:12]

5′ primer - ATATTCTAGATTATACCTGGAATTTATATTCTTCC.

contains a Kozak sequence and a NotI restriction site, and the 3′ primer contains a XbaI restriction site.

Murine SOCS-3 cDNA was amplified by PCR, using the following primer set: 5′ primer-TATAGCGGCCGCGCCACCATGGTCACCCACAGCAA [SEQ ID NO:13]; 3′ primer-ATATAAGCTTTTAAAGTGGAGCATCATACTA [SEQ ID NO:14]. The 5′ primer contains a Kozak sequence and a NotI restriction site, and the 3′ primer contains a HindIII restriction site.

All three SOCS genes are amplified under the same PCR conditions: one cycle at 96° C. for 2 mins then 35 cycles of 96° C. for 10 seconds, 55° C. for 10 seconds and 72° C. for 1 minute.

PCR products are cloned into the adenovirus shuttle vector, pShuttle-CMV, (6) by standard ligation reactions. Generation of recombinant adenovirus plasmids by homologous recombination is then carried out in the E. coli strain BJ5183 (6). 1 μg of pShuttle-CMV (containing selected SOCS gene) was linearized with PmeI restriction enzyme and purified with a DNA purification kit (Qiagen), then mixed with 100 ng of the adenovirus backbone plasmid; pAdEasy-1. The DNA was then electroporated into E.coli BJ5183, which was then plated out onto LB-agar plates containing 30/g/ml of kanamycin and left at 37° C. for 18 hrs. The smallest colonies were picked and grown in 2 ml LB broth containing 30 μg/ml of kanamycin and placed at 37° C. for 8 hrs. Adenovirus plasmid DNA was extracted from each culture and was screened for the presence of recombinant adenoviral DNA by restriction enzyme digestion in comparison with pAdEasy-1. Direct sequencing of the recombinant adenovirus DNA clones confirmed the presence of SOCS encoding sequence.

Production of recombinant adenovirus for in vivo studies is carried out in 293 cells (viral E1 transformed). 93 cells are cultured in 25 cm² flasks, in OptiMEM media (Gibco BRL), at 37° C. and 10% CO₂ until they are 70% confluent 4 μg of recombinant adenovirus, digested with the Pac1 restriction enzyme, is transfected into 293 cells with, Lipofectamine (Gibco-BRL), according to the manufacturer's instructions. Cells are left for 7-10 days and then harvested by scrapping cells off the bottom of the flask into PBS. Cells are subjected to 5 cycles of a freeze/thawing, and the supernatant can then be used to infect more 293 cells to build up viral stocks. Cell lysis should be evident in the majority of cells approximately 3 days post infection, and should be harvested as described above.

To purify the recombinant adenovirus, the infected 293 cells are harvested and spun at 7000 g 4° C. for 10 minutes. The supernatant is discarded and the cells are resuspended in 10 ml of PBS and subject to 5 cycles of a freeze/thawing. The recombinant adenovirus is then purified through a CsCl gradient, comprising two layers of 1.5 ml and 2.5 ml at densities of 1.45 g/ml and 1.25 g/ml respectively. The CsCl is made-up in 5 mM Tris Cl, 1 mM EDTA pH 7.8. The CsCl gradient containing the recombinant adenovirus spun at 90,000 g for 2 hrs and the virus fraction collected with a 19-gauge needle.

The adenovirus is subject to a second round of CsCl purification. The adenovirus is diluted in CsCl solution at a density of 1.33 g/ml and centrifuged at 105 g for 18 hrs. The adenovirus is recovered with a 19-gauge needle and then placed through a G-25 Sephadex column (Amersham) and the virus fractions collected in PBS containing 10% glycerol. The recombinant adenovirus can then be stored at −70° C. until ready for use.

### EXAMPLE 2

### Adenovirus Expressing SOCS-1 have a Beneficial Therapeutic Effect in a Mouse Model of Rheumatoid Arthritis

Collagen-induced arthritis (CIA) is a model of chronic arthritis that is induced following intradermal immunization of mice with collagen in Complete Freund's Adjuvant. It affects articular joints and is characterized by synovial hyperplasia and inflammation, pannus formation and progressive cartilage and bone degradation. The importance of individual cytokines such as GM-CSF and TNFα in CIA has been extensively studied by antibody neutralisation in vivo over the course of disease or by initiating disease in cytokine gene knockout mice.

For induction of CIA, type II collagen (of bovine or chick origin for example) is dissolved to a concentration of 2 mg/ml in 10 mM acetic acid (overnight at 4° C.) then emulsified in an equal volume of Complete Freunds Adjuvant. Male DBA/1 mice are injected intradermally at several sights into the base of the tail with a total of 100 microliters of the emulsion containing 100 micrograms of collagen. On day 21 mice are given an intraperitoneal booster injection of 100 microgram of type 11 collagen dissolved in phosphate buffered saline with onset of arthritis occurring at around day 25-28.

Just prior to expected onset of CIA, mice are scored visually for appearance of arthritis. Mice without macroscopic signs of arthritis in their paws are selected for treatment groups. Alternatively, to study the impact of treatment on existing disease, mice can be left for longer and those that develop overt arthritis selected for treatment groups.

For treatment selected mice are anaesthetized and a small incision in the skin of the knee joint is performed for the intra-articular injection procedure. Intra-articular injection is performed with 10⁷/6 microlitre of either a SOCS-1 (or other SOCS protein) expressing or an empty or β-galactosidase expressing control recombinant adenovirus. At days 1, 5, 10 and 20 after treatment mice are sacrificed and the skin of the knee joint removed. The appearance of arthritis was assessed and severity score was recorded as per routine methods described elsewhere (7). For histological assessment whole knee joints are removed, fixed, decalcified and paraffin embedded Tissue sections are stained with hematoxylin and eosin and evaluated without knowledge of the treatment groups. Histological changes can be scored according to standard methods. For example, infiltration of cells is scored on a scale of 0-3, depending on the amount of inflammatory cells in the synovial cavity (exudate) and synovial tissue (infiltrate). A characteristic parameter in CIA is the progressive loss of bone. This destruction can be graded on a scale of 0-3, ranging from no damage to complete loss of bone structure. Additional analysis may encompass, for example, immunohistological determination of other cell surface/tissue specific markers of disease progression and severity.

Over-expression of SOCS-1 (or other selected SOCS proteins) within the joint may decrease both incidence and severity of CIA and his may be reflected in histological analysis where cellular-accumulation within the joint and/or the level of bone and cartilage destruction is significantly ameliorated

### EXAMPLE 3

### Analysis of Arthritis in an Animal Model Demonstrates a Regulatory Role for SOCS-1 and Supports the Use of SOCS-Based Gene Therapy for the Treatment of Human Inflammatory Disease

Genetically modified mice with a targeted deletion of the SOCS-1 gene (SOCS-1^{-/-}) die within 3 weeks of birth. The primary mediator of this lethal phenotype is interferon-γ. SOCS-1^{-/-} animals crossed onto an IFN-γ^{-/-} background survive as do SOCS-1^{-/-} treated with an antibody that inhibits IFN-γ activity. SOCS-1-^{-/-}IFN-γ^{-/-} mice are ideal for studying the role of SOCS-1 in the development of various disease pathologies. In the present example, the role of SOCS-1 in regulating the activity of the pr-inflammatory cytokines responsible for the development of arthritis was assessed,

SOCS-1^{+/+} IFN-γ^{-/-} and SOCS-1^{-/-} IFN-γ^{-/-} mice were anaesthetiZed and injected intra-articularly into the knee joint with 10 id of a 20 mg/ml solution of methylated bovine serum albumin (mBSA). At the same time, mice were also injected with 250 ng recombinant human IL-1β subcutaneously into the rear footpad The IL-1 injection was repeated on the next 2 days. The mice were sacrificed on day 7 and the knee joints fixed in 10% v/v neutral buffered formalin for at least 2 days, decalcified and embedded in pal Frontal sections of the knee joints were cut at 4 depths, approximately 100 μm apart and stained with haemotoxylin and eosin.

Assessment of Arthritis:

Joint pathology was assessed in a blinded manner and 5 parameters of arthritis were graded for severity from 0 (normal) to 5 (severe). Exudate was scored according to the presence and relative numbers of inflammatory cells and fibrin-like debris in the joint space. Synovitis was defined as thickening of the synovial lining layer and soft tissue inflammation in the infrapatellar fat pad, joint capsule and the area adjacent to the periosteal sheath Pannus was defined as the encroachment of hyperplastic synovium over the articular surface or at the cartilage-bone junction. Cartilage degradation was evaluated on patellofemoral and tibiofemoral articular surfaces. Done degradation was evaluated as the extent and depth of subchondral and periosteal bone erosion. The Mann-Whitney 2-sample rank test was used to compare mean histologic scores of test and control groups.

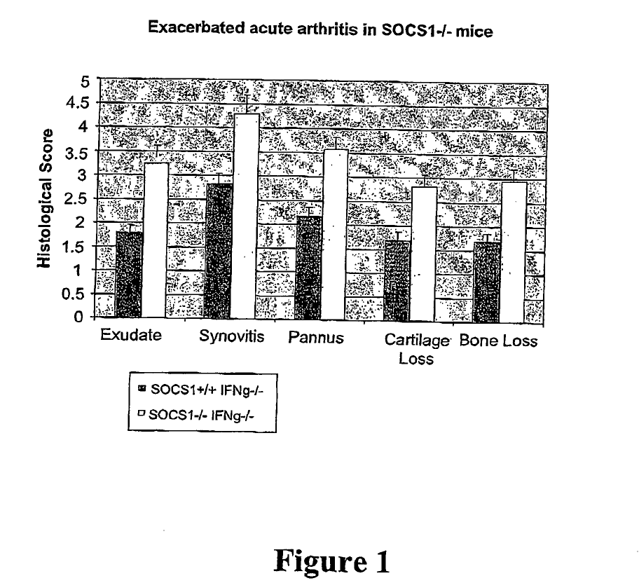

The results demonstrate a role for SOCS-1 in down-regulating/controlling the development of arthritis, in this model of the disease. SOCS-1^{-/-}IFN-γ^{-/-} animals develop more severe arthritis gm control SOCS-1^{+/+}IFN-γ^{-/-} animals (FIG. 1). The severity of the disease in the SOCS-1^{+/+}IFN-γ^{-/-} animals was identical to that routinely observed in wildtype controls (not shown) indicating that the lack of functional SOCS-1 and not INF-γ was responsible for the exacerbation in disease phenotype. Given the clearly demonstrated role for SOCS-1 in the negative regulation of cytokine signalling it is assumed that the exacerbation of disease is the result of the increased activity of proinflammatory cytokines. Over-expression of SOCS-1, following SOCS-1 based gone therapy would inhibit pro-inflammatory cytokine activity and thus ameliorate disease pathology.

The results are shown in tabular form in Table 1 and graphically in FIG. 1.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1

| | | | |
|---|---|---|---|
| Exudate | Synovitis | Pannus | Cartilage loss Bone loss |

| | | | | | | |
|---|---|---|---|---|---|---|
| 2980 | 3 | 3.75 | 2.5 | 2.5 | 2.5 | 14.25 |
| 2981 | 3.33 | 4.67 | 2.33 | 2 | 1.67 | 14 |
| 2982 | 3 | 4 | 3.25 | 2.5 | 3.25 | 16 |
| 2983 | 3 | 3.75 | 3 | 2.5 | 2.75 | 15 |
| 2984 | 3 | 3 | 2.5 | 2.75 | 2 | 13.25 |
| 2985 | 3.25 | 3.5 | 2 | 1.25 | 1 | 11 |
| Average | 3.096666667 | 3.77833333 | 2.59666667 | 2.25 | 2.195 | 13.9166667 |
| Std. | 0.062003584 | 0.22576413 | 0.18577166 | 0.2236068 | 0.32943133 | 0.69721669 |
| Dev. | | | | | | |
| 2986 | 2 | 2.25 | 1.25 | 1 | 1.25 | 7.75 |
| 2987 | 2 | 3 | 3 | 2.25 | 2.75 | 13 |
| 2988 | 1 | 2 | 1.75 | 2 | 1.25 | 8 |
| 2989 | 2 | 4 | 2.75 | 2 | 2 | 12.75 |
| 2990 | 2 | 3.75 | 1.75 | 15 | 2 | 11 |
| 2991 | 1.5 | 2.5 | 2.75 | 2.5 | 2 | 11.25 |
| 2992 | 2.5 | 3 | 2 | 1.25 | 1.75 | 10.5 |
| 2993 | 1 | 2 | 2 | 1 | 1.5 | 7.5 |
| 2994 | 2 | 2.75 | 2.75 | 2 | 1.5 | 11 |
| 2995 | 2 | 3 | 1.75 | 15 | 1 | 9.25 |
| Average | 1.8 | 2.825 | 2.175 | 1.7 | 1.7 | 10.2 |
| Std. Dev | 0.152752523 | 0.21424934 | 0.18652524 | 0.16583124 | 0.16158933 | 0.63113654 |
| 2996 | 4 | 4.75 | 3.5 | 2.75 | 2.5 | 17.5 |
| 2997 | 2.5 | 4 | 4 | 2.5 | 3.5 | 16.5 |
| 2998 | 4 | 5 | 4 | 3.5 | 3.5 | 20 |
| 2999 | 4 | 5 | 4 | 3.25 | 3.25 | 19.5 |
| 3000 | 3 | 4.5 | 3.5 | 3 | 3 | 17 |
| 3001 | 2 | 2.5 | 2.5 | 2 | 2 | 11 |
| Average | 3.25 | 4.29166667 | 3.58333333 | 2.83333333 | 2.95833333 | 16.9166667 |
| Std. Dev | 0.359397644 | 0.3895332 | 0.23863035 | 0.22047928 | 0.24509069 | 1.31286371 |
| Ttest | 0.000736214 | 0.0028622 | 0.00038333 | 0.00099983 | 0.0005242 | 0.00013435 |

### BIBLIOGRAPHY

1. Moriata et al., PNAS 97: 5405-5410, 2000.

2. Altschul et al., Nucl. Acids Res. 25:3389.1997.

3. Ausubel et al., “Current Protocols in Molecular Biology” John Wiley & Sons Inc, 1994-1998, Chapter 15.

4. Bonner and Laskey, Eur. J. Biochem. 46: 83, 1974.

5. Marmur and Doty, J. Mol. Biol. 5: 109, 1962.

6. He et al., PNAS 95: 2509-2514, 1998.

7. Campbell et al., Annals. Rheum. Dis. 56: 364-368, 1997.

1. A method for modulating cytokine or hormone signaling in an animal, said method comprising regulating expression of a genetic sequence encoding a SOCS protein or its derivative or homolog in said animal.

2. A method according to claim 1 wherein the SOCS protein comprises a SOCS box in its C-terminal regional comprising the amino acid sequence:—
X₁X₂X₃X₄X₅X₆X₇X₈X₉X₁₀X₁₁X₁₂X₁₃X₁₄X₁₅X₁₆[Xᵢ]₁₇X₁₈X₁₉X₂₀X₂₁X₂₂X₂₃[Xⱼ]ₙX₂₄X₂₅X₂₆X₂₇X₂₈
wherein
X₁ is L, I, V, M, A or P;
X₂ is any amino acid residue;
X₃ is P, T or S;
X₄ is L, I, V, M, A or P;
X₅ is any amino acid;
X₆ is any amino acid;
X₇ is L, I, V, M, A, F, Y or W;
X₈ is C, T or S;
X₉ is R, K or R;
X₁₀ is any amino acid;
X₁₁ is any amino acid;
X₁₂ is L, I, V, M, A or P;
X₁₃ is any amino acid;
X₁₄ is any amino acid;
X₁₅ is any amino acid;
X₁₆ is L, I, V, M, A, P, G, C, T or S;
[Xᵢ]ₙ is a sequence of amino acids wherein n is from 1 to 50 amino acids and wherein the sequence Xᵢ may comprise the same or different amino acids selected from any amino acid residue;
X₁₇ is L, I, V, M, A or P;
X₁₈ is any amino acid;
X₁₉ is any amino acid;
X₂₀ is L, I, V, M, A or P;
X₂₁ is P;
X₂₂ is L, I, V, M, A, P or G;
X₂₃ is P or N;
[Xⱼ]ₙ is a sequence of n amino acids wherein n is from 0 to 50 amino acids and wherein the Xⱼ may comprise the same or different amino acids selected from any amino acid residue;
X₂₄ is L, I, V, M, A or P;
X₂₅ is any amino acid;
X₂₆ is any amino acid;
X₂₇ is Y or F;
X₂₈ is L, I, V, M A or P.

3. A method according to claim 1 or 2 wherein the SOCS protein comprises a protein:molecule interacting region N-terminal of a SOCS box.

4. A method according to claim 3 wherein the protein:molecule interacting region is selected from an SH2 domain, WD-40 repeats and ankyrin repeats.

5. A method according to claim 1 wherein the animal is a human, primate, livestock animal, laboratory test animal or a companion animal.

6. A method according to claim 5 wherein the animal is a human.

7. A method according to claim 1 wherein the hormone is a protein or non-protein hormone.

8. A method according to claim 7 wherein the hormone is selected from a growth hormone, insulin-like growth factor-I or prolactin.

9. A method according to claim 8 wherein the hormone is growth hormone.

10. A method according to claim 1 wherein the cytokine is an interleukin, tumor necrosis factor, a colony stimulating factor or an interferon.

11. A method according to claim 1 comprising overexpression of said genetic sequence encoding said SOCS protein.

12. A method according to claim 11 wherein the SOCS protein is SOCS-1.

13. A method according to claim 12 wherein the SOCS protein is human SOCS-1.

14. A method according to claim 11 or 12 wherein over-expression of the genetic sequence is via an expression vector.

15. A method according to Claim 14 wherein the expression vector is a viral vector.

16. A method according to claim 15 wherein the viral vector is an adenovirus, adeno-associated virus or retrovirus.

17. A method according to claim 14 wherein the expression vector is a plasmid-based vector.

18. A method of modulating cytokine or hormone signalling in an animal and in particular a human, said method comprising up-regulating expression of a genetic sequence encoding a SOCS protein or increasing in said animal and wherein said SOCS protein comprises a protein:molecule interaction region such as but not limited to an SH2 domain, WD-40 repeats and/or ankyrin repeats, N-terminal of a SOCS box, wherein said SOCS box comprises the amino acid sequence:—
X₁X₂X₃X₄X₅X₇X₈X₉X₁₀X₁₁X₁₂X₁₃X₁₄X₁₅X₁₆[Xᵢ]ₙX₁₇X₁₈X₁₉X₂₀X₂₁X₂₂X₂₃[Xⱼ]ₙX₂₄X₂₅X₂₆X₂₇X₂₈
wherein:
X₁ is L, I, V, M, A or P;
X₂ is any amino acid residue;
X₃ is P, T or S;
X₄ is L, I, V, M, A or P;
X₅ is any amino acid;
X₆ is any amino acid;
X₇ is L, I, V, M, F, Y or W;
X₈ is C, T or S;
X₉ is R, K or H;
X₁₀ is any amino acid;
X₁₁ is any amino acid;
X₁₂ is L, I, V, M, A or P,
X₁₃ is any amino acid;
X₁₄ is any amino acid;
X₁₅ is any amino acid;
X₁₆ is L, I, V, M, A, P, G, C, T or S;
[Xᵢ]ₙ is a sequence of n amino acids wherein n is from 1 to 50 amino acids and wherein the sequence Xᵢ may comprise he same or different amino acids selected from any amino acid residue;
X₁₇ is L, I, V, M, A or P;
X₁₈ is any amino acid;
X₁₉ is any amino acid;
X₂₀ is L, I, V, M, A or P;
X₂₁ is P;
X₂₂ is V, M, A, P or G;
X₂₃ is P or N;
[Xⱼ]ₙ is a sequence of n amino acids wherein n is from 0 to 50 amino acids and wherein the Xⱼ may comprise the same or different amino acids selected from any amino acid residue;
X₂₄ is L, I, V, M, A or P;
X₂₅ is any amino acid;
X₂₆ is any amino acid,
X₂₇ is Y or F;
X₂₈ is L, I, V, M, A or P.

19. A method according to claim 18 wherein the hormone is a protein or non-protein hormone.

20. A method according to claim 19 wherein the hormone is selected from a growth hormone, insulin-like growth factor-I or prolactin.

21. A method according to claim 20 wherein the hormone is growth hormone.

22. A method according to claim 18 wherein the cytokine is au interleukin, tumor necrosis factor, a colony stimulating factor or an interferon.

23. A method according to claim 18 comprising overexpression said genetic sequence encoding said SOCS protein.

24. A method according to claim 1 or 18 wherein the SOCS protein comprises an amino acid sequence selected from SEQ ID NO:2, SEQ ID NO:4, SEQ ID NO:6 or SEQ ID NO:8 or an amino acid sequence having at least about 60% similarity after optimal alignment to SEQ ID NO:2, SEQ ID NO:4, SEQ ID NO:6 or SEQ ID NO:8.

25. A method according to claim 1 wherein the SOCS protein is encoded by the nucleotide sequence set forth in SEQ ID NO: 1, SEQ ID NO:3, SEQ ID NO:5 or SEQ ID NO:7 or a nucleotide sequence having at least about 60% similarity to SEQ ID NO: 1, SEQ ID NO:3, SEQ ID NO:5 or SEQ ID NO:7 or a nucleotide sequence capable of hybridizing to SEQ ID NO:1, SEQ ID NO:3, SEQ ID NO:5 or SEQ ID NO:7 or their complements under low stringency conditions.

26. A method according to claim 1 wherein the SOCS protein is human SOCS-1, murine SOCS-1 or rat SOCS-1.

27. A method according to claim 18 wherein the SOCS protein is SOCS-1.

28. A method according to claim 27 wherein the SOCS protein is human SOCS-1.

29. A method according to claim 18 wherein up-regulating expression of the SOCS genetic sequence is via an expression vector.

30. A method according to claim 29 wherein the expression vector is a viral vector.

31. A method according to claim 30 wherein the viral vector is an adenovirus, adeno-associated virus or retrovinis.

32. A method according to Claim 29 wherein the expression vector is a plasmid-based vector.

33. A genetically modified animal having a mutation in one or both alleles of SOCS-1.

34. A genetically modified animal according to claim 33 wherein the animal is a mouse.

35. A genetically modified animal according to claim 33 or 34 carrying a deletion mutation in one or both alleles encoding SOCS-1.

36. Use of an expression vector encoding a SOCS protein to facilitate over-expression of said SOCS protein in an animal cell.

37. Use according to claim 36 wherein the animal cell is a human cell.
